(19) United States
(12) Patent Application Publication
(10) Pub. No.: US 2019/0076192 A1
(43) Pub. Date: Mar. 14, 2019

(54) METHOD AND SYSTEM FOR CONFIGURING BIOMEDICAL LASER

(71) Applicant: Modulight Oy, Tampere (FI)

(72) Inventors: Ville Vilokkinen, Tampere (FI); Petteri Uusimaa, Tampere (FI); Seppo Orsila, Ylöjärvi (FI)

(21) Appl. No.: 15/703,238

(22) Filed: Sep. 13, 2017

(51) Int. Cl. A61B 18/20; H01S 3/10; H01S 3/13; G06F 19/00

(52) U.S. Cl. CPC A61B 18/20; H01S 3/10069; A61B 2017/00022; G06F 19/3481; H01S 3/1305

(57) ABSTRACT

A method and a system for configuring a first biomedical laser. The method includes providing a first set of operational parameters; operating the first biomedical laser using the first set of operational parameters; measuring an operational response of the first biomedical laser; measuring a light output properties of the first biomedical laser; analyzing the measured light output properties and the operational response; and triggering an action based on the analysis.

### TECHNICAL FIELD

The present disclosure relates generally to use of lasers for biomedical applications; and more specifically, to methods for configuring biomedical lasers. Furthermore, the present disclosure also relates to systems for configuring aforementioned biomedical lasers.

### BACKGROUND

Nowadays, lasers are increasingly being used for several biomedical applications. Though prevalent in ophthalmology and dermatology, lasers have also found use in surgical procedures, cancer diagnosis and treatment, biomedical imaging, gene sequencing and the like. Therefore, in many biomedical applications, lasers have become a mainstay and are quickly replacing conventional tools. This is possible since operational parameters (such as wavelength of emitted light, intensity of the emitted light, and so forth) of lasers can be configured for various biomedical applications. Often, a laser is configured to emit light of a wavelength that matches absorption lines of a certain type of body tissue or chemical compound like drug or organic molecule like base in DNA, thereby triggering a specific biochemical or biomechanical process in the target. For example, a certain wavelength of light affects only the matching body tissue while having no effect on other body tissues. For example, a carbon dioxide gas laser can be used for laser surgery on soft tissue, whereas dye lasers are best suited for dermatological applications since wavelength of the dye lasers matches absorption lines of tissues including melanin or hemoglobin.

However, due to high risk and precision requirements associated with biomedical applications, lasers used in medicine (or biomedical lasers) are required to operate within predefined operational parameter ranges. Therefore, as an example, a biomedical laser for surgery, therapy or a specific biochemical activation may be required to always emit a specific wavelength of light with a specific output power, for a precise amount of time. Furthermore, deviation of the operational parameters of biomedical lasers to lie outside of typically approved tolerance ranges thereof may cause severe, and in some cases fatal, injuries or cause wrong biomedical diagnosis or analysis. Additionally, light emitting sources and other components (such as optical couplers, light guides, and the like) of the biomedical lasers may often deteriorate or break down, consequently leading to difficulties in operation thereof.

Furthermore, present techniques for maintaining configuration of the biomedical lasers often require frequent calibration of the biomedical lasers to maintain a desired intensity and wavelength of the emitted light with respect to a specific current and voltage input. However, such techniques often fail to monitor and control the biomedical lasers during regular operation thereof. As an example, fluctuations in the specific current and voltage input to the biomedical lasers may not be detected during operation thereof, consequently adversely impacting configuration of the biomedical lasers. Therefore, performance of the biomedical lasers may be sub-optimal or even harmful.

Therefore, in light of the foregoing discussion, there exists a need to overcome the aforementioned drawbacks associated with biomedical lasers.

### SUMMARY

The present disclosure seeks to provide a method for configuring a first biomedical laser. The present disclosure also seeks to provide a system for configuring a first biomedical laser. The present disclosure seeks to provide a solution to the existing problems of inaccuracies in configuring biomedical lasers, and difficulties in maintaining the configuration of the biomedical laser. An aim of the present disclosure is to provide a solution that overcomes at least partially the problems encountered in prior art, and provides reliable and efficient techniques for configuring biomedical lasers.

In one aspect, an embodiment of the present disclosure provides a method for configuring a first biomedical laser, the method comprising
providing a first set of operational parameters;
operating the first biomedical laser using the first set of operational parameters;
measuring an operational response of the first biomedical laser;
measuring a light output properties of the first biomedical laser;
analyzing the measured light output properties and the operational response; and
triggering an action based on the analysis.

In another aspect, an embodiment of the present disclosure provides a system for configuring a first biomedical laser, the system comprising
a processor coupled to the first biomedical laser, the processor being configured to
provide a first set of operational parameters;
operate the first biomedical laser using the first set of operational parameters;
measure an operational response of the first biomedical laser; and
a light sensing arrangement communicably coupled to the processor, the light sensing arrangement being configured to measure a light output properties of the first biomedical laser;

wherein the system is further configured to analyze the measured light output properties and the operational response, and trigger an action based on the analysis.

Embodiments of the present disclosure substantially eliminate or at least partially address the aforementioned problems in the prior art, and enables easy, optimal configuration and monitoring of biomedical lasers.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a system for configuring a first biomedical laser, in accordance with an embodiment of the present disclosure;

FIG. 2 illustrates a schematic block diagram of a processor in communication with a server arrangement, in accordance with an embodiment of the present disclosure;

FIG. 3 illustrates steps of a method for configuring a first biomedical laser, in accordance with an embodiment of the present disclosure; and

FIG. 4 is a schematic illustration of a system for configuring a first biomedical laser, in accordance with another embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a method for configuring a first biomedical laser, the method comprising
providing a first set of operational parameters;
operating the first biomedical laser using the first set of operational parameters;
measuring an operational response of the first biomedical laser;
measuring a light output properties of the first biomedical laser;
analyzing the measured light output properties and the operational response; and
triggering an action based on the analysis.

In another aspect, an embodiment of the present disclosure provides a system for configuring a first biomedical laser, the system comprising
a processor coupled to the first biomedical laser, the processor being configured to
provide a first set of operational parameters;
operate the first biomedical laser using the first set of operational parameters;
measure an operational response of the first biomedical laser; and
a light sensing arrangement communicably coupled to the processor, the light sensing arrangement being configured to measure a light output properties of the first biomedical laser;

wherein the system is further configured to analyze the measured light output properties and the operational response, and trigger an action based on the analysis.

The present disclosure provides the aforementioned method and the aforementioned system for configuring the first biomedical laser. The method described herein is easy to implement and allows for accurate configuration of the first biomedical laser. Therefore, the first biomedical laser is efficiently operable to emit light within predefined operational parameter ranges. Beneficially, the disclosed method and system enable the monitoring and control of the first biomedical laser during its operation. As an example, transients (or fluctuations) in input parameters of the first biomedical laser may be detected during its operation. Beneficially, such timely and efficient control of the first biomedical laser enhances performance thereof, and reduces frequency or allows timely scheduling of required calibration or servicing of the first biomedical laser.

Throughout the present disclosure, the term ‘first biomedical laser’ relates to a device (or an equipment) employing use of a laser light emitter to generate light for purpose of biomedical applications (namely biomedical diagnosis and biomedical procedures). Examples of such biomedical applications include, but are not limited to, cosmetic procedures, surgical procedures, biomedical imaging or illumination, drug activation, and dental or ophthalmic treatments. Therefore, the first biomedical laser is required to be configured differently for different biomedical applications.

It will be appreciated that the first biomedical laser may be configured for at least one biomedical application. Optionally, in this regard, the first biomedical laser is associated with at least one treatment modality. Throughout the present disclosure, the term ‘treatment modality of the first biomedical laser’ relates to at least one mode (or at least one manner) of operation of the first biomedical laser, wherein such at least one mode of operation depends on the at least one biomedical application. As an example, the first biomedical laser may be configured for two biomedical applications, namely photodynamic therapy and photocoagulation. In such an example, the first biomedical laser may be associated with two treatment modalities M1 and M2, wherein the treatment modality M1 relates to photodynamic therapy and the treatment modality M2 relates to photocoagulation.

As mentioned previously, the first set of operational parameters is provided. Throughout the present disclosure, the term ‘first set of operational parameters’ relates to a set of factors that define conditions of operation of the first biomedical laser. Optionally, the first set of operational parameters comprises at least one of a target wavelength of the first biomedical laser, a target energy output of the first biomedical laser and an operational mode selected from a group of a treatment modality, biomedical activation and measurement process of the first biomedical laser. It will be appreciated that the first set of operational parameters are directly related to the at least one biomedical application for which the first biomedical laser may be configured.

Additionally, optionally, the first set of operational parameters further comprises at least one of current input for operation of the first biomedical laser, voltage input for operation of the first biomedical laser and operating temperature of the first biomedical laser.

Furthermore, optionally, the at least one treatment modality of the first biomedical laser comprises at least one of the target wavelength of the first biomedical laser, the target energy output of the first biomedical laser, the current input for operation of the first biomedical laser, the voltage input for operation of the first biomedical laser and the operation temperature of the first biomedical laser. In such an instance, providing the treatment modality of the first biomedical laser may automatically provide at least one operational parameter for operation of the first biomedical laser.

Referring to an aforementioned example wherein the first biomedical laser is associated with the two treatment modalities M1 and M2, the treatment modality M1 may comprise a target wavelength of X1 nanometer and E1 target energy output whereas the treatment modality M2 may comprise a target wavelength of X2 nanometer and E2 target energy output. In such an instance, providing the treatment modality M1 may automatically provide the target wavelength and target energy output operational parameters of the first biomedical laser. Beneficially, such biomedical laser may be configured to support multiple application or modalities that can be activated remotely by calling the operational parameters of each application.

Optionally, the first set of operational parameters is provided by a trained biomedical practitioner. Optionally, in this regard, the trained biomedical practitioner may input the first set of operational parameters at a computing device including the processor coupled to the first biomedical laser.

As mentioned previously, the first biomedical laser is operated using the first set of operational parameters. It will be appreciated that the processor controls operation of the laser light emitter to emit light in accordance with the first set of operational parameters. In such an instance, the processor provides the current input and the voltage input and the temperature input for operation of the first biomedical laser, to the laser light emitter.

In an embodiment, the first biomedical laser is operated continuously using the first set of operational parameters. In such an instance, the first biomedical laser is operated to emit light without interruption, for a predetermined period of time. Optionally, in such an instance, the first set of operational parameters is constant over the predetermined period of time.

In another embodiment, the first biomedical laser is operated as a series of pulses using the first set of operational parameters. In such an instance, the first biomedical laser is operated to emit light at periodic time instants. Furthermore, a time duration between two periodic time instants may comprise a first time period and a second time period, wherein the first biomedical laser is operable to emit light during the first time period and the first biomedical laser is switched off during the second time period. Beneficially, the first biomedical laser may be operated as the series of pulses for soft tissue surgery, for prevention of necrosis or other unwanted tissue deformation. It will be appreciated that emission of light in such a pulsed manner prevents overheating of tissues surrounding a target tissue by allowing for the tissues to cool down during the second time duration (when the first biomedical laser is switched off). In another application the pulsing of light may ensure efficient drug activation or resulting treatment affect through a precise timing of a biochemical process.

Optionally, the laser light emitter comprises at least one light emitting element, such as a light emitting circuit, wherein the processor is configured to control the at least one light emitting circuit to emit light of at least one target wavelength. As an example, the laser light emitter of the first biomedical laser may be operated to emit monochromatic light at a target wavelength of 632 nanometer for activating a specific drug or dye molecule in photodynamic therapy application. In such an example, optionally, the same laser light emitter of the first biomedical laser may be operated to emit monochromatic light at a target wavelength of 635 nanometer for activating another drug or dye molecule in photodynamic therapy. In another example, the laser light emitter of the first light emitting circuit of the first biomedical laser may be operated to emit monochromatic light at a target wavelength of 532 nanometer for activating a fluorescent dye in a gene sequencing application, whereas the second light emitting circuit of the first biomedical laser may be operated to emit a second wavelength of 660 nm for activating another set of fluorescent dyes in a gene sequencing application, and the second light emitting circuit may be activated for use at a later period of time also remotely. The biomedical laser can comprise a plurality of light emitting circuits. The light emitting circuits may be operated to emit monochromatic light with same or different target wavelengths. The circuits might be independently operated from each others. For example a first light emitting circuit might emit a target wavelength of 532 nanometers and a second might emit an other target wavelength of 660 nanometers. As another example the first light emitting circuit might provide light continuously and the second might provide it in pulsed matter during a treatment modality.

As mentioned previously, the operational response of the first biomedical laser is measured. It is to be understood that the term ‘operational response of the first biomedical laser’ relates to variation of operational characteristics of the first biomedical laser, whilst the first biomedical laser is operated using the first set of operational parameters. Optionally, measuring the operational response of the first biomedical laser comprises measuring at least one of voltage transient as a function of time, current transient as a function of time, temperature of the laser light emitter and power usage of the first biomedical laser during operation. Furthermore, it will be appreciated that operational response parameters are measured by employing measuring instruments such as voltage meters, current meters, thermometers, and the like. As an example, the voltage transient (namely voltage fluctuations within the laser light emitter) and the current transient (namely current fluctuations within the laser light emitter) may be measured as a function of time, to constitute measured operational response of the first biomedical laser. In such an example, the processor coupled to the first biomedical laser may record variation of the current input and the voltage input with regard to time to detect and measure the current and voltage transients respectively.

Optionally, the operational response of the first biomedical laser is measured during an entire time duration of operation thereof. As an example, for a first biomedical laser ML1 that is operated continuously, the operational response may be measured during an entire predetermined period of time for which the first biomedical laser emits light. Alternatively, optionally, the operational response of the first biomedical laser is measured during at least a part of the entire time duration of operation of the first biomedical laser. In an example, the operational response of the first biomedical laser ML1 may be measured during first fifteen minutes of operation thereof. In another example, for a first biomedical laser ML2 that is operated as a series of pulses, the operational response may be measured only during the first time period of light emission from the first biomedical laser ML2.

Furthermore, as mentioned previously, the light output properties of the first biomedical laser is measured. It is to be understood that the term ‘light output properties of the first biomedical laser’ relates to properties (namely characteristics) of the light emitted from the first biomedical laser. Optionally, measuring the light output properties of the first biomedical laser comprises measuring at least one of a wavelength of the emitted laser light, a spectrum of the emitted laser light, an energy of the emitted laser light, a pulse energy of the emitted laser light, an optical power of the emitted laser light, and a pulse form factor of the emitted laser light. It will be appreciated that measurement of the light output properties of the first biomedical laser is performed whilst the first biomedical laser is switched on to emit light therefrom.

Optionally, measuring the light output properties is carried out by arranging at least one of a first light sensor in a laser component module, a second light sensor in proximity of a light guide, a third light sensor at an end of the light guide and a fourth light sensor in an optical head. Specifically, the emitted laser light (from the first biomedical laser) may be incident on light-sensitive elements of the first, second, third and fourth light sensors. It is to be understood that an arrangement of the first light sensor, the second light sensor, the third light sensor and the fourth light sensor collectively constitute the light sensing arrangement configured to measure the light output properties of the first biomedical laser. Beneficially, the aforesaid arrangement of the first, second, third and fourth light sensors measures the light output properties at multiple positions within the first biomedical laser, thereby allowing for detection of dissimilarities in the light output properties that may occur at the multiple positions. It is also possible to arrange less or more than four light sensors, such as one, two, three, five, six or seven light sensors. By the term “proximity” it is means a position where the light emitted can effectively be measured.

In an exemplary implementation, the first light sensor may be arranged in the laser component module to measure ambient light within the laser component module. In this regard, the laser component module may include the laser light emitter arranged therein, wherein the laser light emitter may be optically coupled to the light guide via a light coupling arrangement. In such an instance, at least a part of the light guide may lie within the laser component module. Therefore, the first light sensor may be operable to measure intensity (or energy) of the ambient light that results from at least one of: leakage of the emitted laser light from the laser light emitter, leakage of the emitted laser light from the light coupling arrangement, leakage of the emitted laser light from the part of the light guide within the laser component module, leakage of the emitted laser light from an interface between the laser light emitter and the light coupling arrangement and leakage of the emitted laser light from an interface between the light coupling arrangement and the light guide. It will be appreciated that in this regard, a high value of measured intensity of the ambient light indicates a high magnitude of leakage of the emitted laser light, which may be undesirable.

Furthermore, in such an exemplary implementation, the second light sensor may be arranged in proximity of the light guide to measure the energy of the emitted laser light that may leak from the light guide. In such an instance, the second light sensor may be attached, for example, to the light guide. Moreover, in such an exemplary implementation, the third light sensor may be arranged at an end of the light guide, from which the emitted laser light may be administered for the purpose of biomedical applications. In such an instance, the third light sensor may be configured to measure the wavelength and the energy of the emitted laser light. In further exemplary implementation, the fourth light sensor may be arranged in an optical head. The optical head is a component or element that might be optionally arranged at the end of the light guide. The optical head and the fourth sensor can be thus configured to a contained space, where a collimated laser beam can travel and be further manipulated and monitored.

As mentioned previously, the measured light output properties and the operational response are analyzed. It will be appreciated that such analysis may be performed for identification of undesired errors (namely operational deviations) in operation of the first biomedical laser. Optionally, such analysis may also facilitate in predicting possible future occurrence of the undesired errors. Beneficially, such identification of undesired errors may be identified during operation of the first biomedical laser, thereby facilitating prompt and timely control of the first biomedical laser. Optionally, the analysis comprises comparing the measured operational response and the light output properties with a first set of reference values. It will be appreciated that comparison between the aforesaid measured data (namely the measured operational response and the light output properties) and expected data (namely the first set of reference values) allows for identification of undesired errors in operation of the first biomedical laser. Furthermore, identification of significant variation between the measured data and the expected data is indicative of faulty/undesirable operation of the first biomedical laser.

Throughout the present disclosure, the term ‘first set of reference values’ relates to a collection of expected values (or attributes) of the emitted laser light, when the first biomedical laser operates normally (namely as per requirement). Optionally, in this regard, the first set of reference values comprises at least one of a target wavelength of the first biomedical laser, a target energy output and a pulse form factor. As an example, a first set of reference values for a first biomedical laser ML3 may comprise a target wavelength equivalent to 700 nanometer, and a pulse form factor equivalent to 1. Therefore, expected values of emitted laser light from the first biomedical laser ML3, under normal operational conditions, constitute the aforesaid first set of reference values.

Additionally, optionally, the first set of reference values further comprises at least one of a range of the target wavelength, a range of the target energy output and a range of the pulse form factor. Alternatively, the first set of reference values may comprise at least one of a target wavelength, a target energy output and a pulse form factor. In such an instance, the aforesaid ranges relate to acceptable tolerance limits (namely values within a lower threshold value and a higher threshold value) of the expected values of the emitted laser light, when the first biomedical laser operates normally. It is to be understood that upon comparison, if the measured light output properties and the operational response are found to lie outside of the aforesaid ranges, the faulty or undesirable operation of the first biomedical laser is indicated.

In an exemplary scenario, an energy of the emitted laser light and power usage of the first biomedical laser may be measured at two time instants T1 and T2. For example, at both the time instants T1 and T2, the energy of the emitted laser light is measured to be equivalent to E1. However, the power usage of the first biomedical laser at the time instants T1 and T2 may be measured to be equivalent to P1 and P2, wherein P2 is greater than P1. Therefore, analysis of the measured energy of the emitted laser light and power usage of the first biomedical laser may predict possible breakdown of at least one component of the first biomedical laser and thus preventing possible undesired or even harmful use the laser unit.

In another exemplary scenario, a wavelength of emitted laser light may be measured at two time instants T3 and T4. For example, the wavelength of emitted laser light at the time instant T3 is measured to be 470 nanometer whereas the wavelength of emitted laser light at the time instant T4 is measured to be 473 nanometer. In such a scenario, analysis of the measured wavelength of emitted laser light may indicate that the laser light emitter of the first biomedical laser is unstable and highly prone to breakdown. Optionally, it may be concluded to be outside the range of acceptable wavelength range of the specified biomedical application, or it may require recalibration of the optical power or energy to meet again the required spectral energy of the said application.

As mentioned previously, the action is triggered based on the analysis. Throughout the present disclosure, the term ‘action’ relates to at least one response (namely at least one event) pertaining to control of the first biomedical laser, so as to operate the first biomedical laser normally (namely as per requirement). It will be appreciated that the triggered action may pertain to at least one of: implementation of corrective measures in an event of faulty operation of the first biomedical laser, discontinuing use of the first biomedical laser and use of the first biomedical laser for a different or limited treatment modality or indication. Optionally, the triggered action may further pertain to implementation of biomedical research by employing the analysis of the first biomedical laser.

Optionally, the action is deriving a second set of operational parameters based on the analysis and providing the second set of operational parameters. It will be appreciated that the second set of operational parameters may be derived in an event of the faulty or undesirable operation of the first biomedical laser. More optionally, in this regard, the second set of operational parameters comprises operational parameters for the different treatment modality, parameters for biomedical activation or parameters for biomedical measurement process. In such an instance, the first biomedical laser may be operated using the second set of operational parameters for a different or additional biomedical application than an originally intended biomedical application. As an example, a first biomedical laser ML4 may be operated using a first set of operational parameters for an originally intended surgical procedure. In such an example, the first set of operational parameters may comprise a target wavelength of the first biomedical laser ML4 to be equal to 850 nanometer. However, analysis of measured light output properties of the first biomedical laser ML4 may indicate that the first biomedical laser ML4 is unable to emit light of wavelengths greater than 830 nanometer. In such an instance, an action is triggered based on the analysis wherein the action is deriving a second set of operation parameters. For example, the second set of operational parameters may comprise a target wavelength of the first biomedical laser ML4 to be equal to 810 nanometer. Therefore, the second set of operational parameters may be provided to operate the first biomedical laser ML4 for a different treatment modality. Such different treatment modality may relate to operating the first biomedical laser ML4 for dentistry or different surgical modality. It is also possible that the laser energy of the first biomedical laser ML4 may decrease over time and thus limit its use for limited modalities or biomedical processes.

Optionally, the action is initiating a maintenance procedure for the first biomedical laser. It will be appreciated that at least one component of the first biomedical laser may be repaired and/or replaced as part of maintenance procedure. In such an instance, a trained maintenance personnel may implement the aforesaid maintenance procedure for the first biomedical laser thereby eliminating time consuming repair process after failure or minimizing the down time of the biomedical laser use in high throughput biomedical processes like gene sequencing.

Furthermore, optionally, the method further comprises transmitting the measured operational response and the light output properties to a server arrangement via a network. The network can be for example Internet. The server arrangement can be a server system in a cloud or a dedicated server system. Further the analyzing the measured light output properties and the operational response is/can be carried out in the server arrangement. This setup enables to configure and update analysis algorithms in a centralized manner. Further the setup enables a way to collect measured light output properties and the operational responses from plurality of biomedical lasers. Indeed, measured light output properties and operational responses from a first biomedical laser can be used derive operational parameters for a second biomedical laser. The setup further enables to provide parameters to configure a first or second biomedical laser from the server arrangement. The setup further enables to trigger actions such as maintenance from the server arrangement. The server arrangement can be used to provide for example a web interface to access information related to the biomedical lasers. Beneficially, this server arrangement allows for example centralized monitoring of all connected biomedical lasers, switching on and off different functions or modalities, and controlling the use and access of the biomedical lasers in different locations.

Furthermore, optionally, the method further comprises storing the measured operational response and the light output properties, basing a second set of reference values on the stored measured operational response and the light output properties, and using the second set of reference values for configuring a second biomedical laser. It is to be understood that the second set of reference values relates to a collection of expected values (or attributes) of the emitted laser light, when the second biomedical laser operates normally. Therefore, the second biomedical laser may be beneficially configured by keeping into consideration historical operational data of the first biomedical laser.

Optionally, storing the measured operational response and the light output properties comprises storing the measured operational response and the light output properties at a memory unit of the processor coupled to the first biomedical laser. It will be appreciated that the stored operational response and the light output properties may constitute a historical log of the measured data of the first biomedical laser, and such historical log may be beneficially utilized during future use of the first biomedical laser. Furthermore, beneficially, the locally stored historical log of the measured data of the first biomedical laser may be easily accessed by the trained biomedical practitioner or operator. Optionally, in this regard, the stored operational response and the light output properties may further be utilized to facilitate biomedical research that may require the analysis of the first biomedical laser.

More optionally, storing the measured operational response and the light output properties comprises transmitting the measured operational response and the light output properties to a server arrangement via a network and storing the measured operational response and the light output properties at a database of the server arrangement. In such an instance, the network may be for example Internet. Furthermore, optionally, historical logs of operational data of multiple biomedical lasers are stored at the database of the server arrangement. Beneficially, storing the measured operational response and the light output properties may facilitate the trained biomedical practitioner to employ a new use of the first biomedical laser for a previously unknown biomedical application. Further, storing the measured operational response and the light output properties may facilitate the learning algorithms to derive a new use of the first biomedical laser for a previously unknown biomedical application and activating them in use remotely by utilizing the described monitoring of operational parameters and the server arrangement.

According to another embodiment, the method further comprises identifying a person, extracting from the database a profile data of the person and providing the first set of operational parameters based on the profile data. In yet another embodiment, the method further comprises obtaining a location of the biomedical laser, extracting from the database a setting data of the associated with the biomedical laser and the location and providing the first set of operational parameters based on the setting data. The method may still further comprise obtaining product information, extracting from the database an operational data associated with the product information and the biomedical laser and providing the first set of operational parameters based on the operational data.

As mentioned previously, the system for configuring the first biomedical laser comprises the processor coupled to the first biomedical laser. It will be appreciated that the processor is communicably coupled with the first biomedical laser. Furthermore, as mentioned previously, the processor is configured to provide the first set of operational parameters, operate the first biomedical laser using the first set of operational parameters, and measure the operational response of the first biomedical laser. According to embodiments of the present disclosure, the processor may be hardware, software, firmware or a combination of these, operable to facilitate configuration of the first biomedical laser.

Optionally, the processor comprises a memory module, the memory module being configured to store the measured operational response and the light output properties. For example, the memory module may store the measured power usage of the first biomedical laser and/or the measured temperature of the laser light emitter during operation. Examples of the memory module, include but are not limited to, random access memory, hard disk drive, flash memory and optical disc.

Furthermore, optionally, the first biomedical laser comprises the laser light emitter arranged in the laser component module and the light guide optically coupled to the laser light emitter via the light coupling arrangement. In an embodiment, the laser light emitter comprises the at least one light emitting circuit operable to emit light of the at least one target wavelength.

Furthermore, it will be appreciated that the laser component module beneficially provides a casing (or a protective housing) for the laser light emitter of the first biomedical laser. Furthermore, the laser component module may beneficially prevent leakage of the emitted laser light produced by the laser light emitter, into an environment (namely surrounding) of use of the first biomedical laser. Optionally, the laser component module may comprise a cylindrical body coaxially disposed with a direction of emission of the emitted laser light, in a manner that the laser component module completely encloses the laser light emitter. In such an instance, the laser component module may be arranged so as not to interfere (or obstruct) an optical path of the emitted laser light produced by the laser light emitter. Optionally, the laser component module comprises an opening at a surface thereof to allow the light guide to pass through. In such an instance, dimensions of the light guide and the opening may be such that the emitted laser light does not leak into the environment of use of the first biomedical laser.

Throughout the present disclosure, the term ‘light guide’ used herein relates to equipment configured to allow passage of light therethrough. In an embodiment, the light guide is an optical fibre cable. In another embodiment, the light guide is an optical waveguide. The light guide may be optically coupled to the laser light emitter by way of the light coupling arrangement. In such an embodiment, the at least a part of the light guide may lie within the laser component module. As mentioned previously, one end of the light guide is coupled with the light coupling arrangement, and the other end of the light guide is used to administer the emitted laser light for the purpose of biomedical applications. Furthermore, the light guide can comprise an optical head i.e. a contained space, where a collimated laser beam can travel and be further manipulated. The optical head can be arranged at an end of the light guide such as optical fibre.

Additionally, the light coupling arrangement is disposed inside the laser component module. The light coupling arrangement is configured to provide a coupling arrangement between the laser light emitter and the light guide. In an embodiment, the light coupling arrangement detachably couples the laser light emitter and the light guide. Optionally, the light coupling arrangement may be a device that provides a leak proof connection between the laser light emitter and the light guide.

Additionally, as discussed above, the system comprises the light sensing arrangement communicably coupled to the processor. As discussed previously, the light sensing arrangement is configured to measure a light output properties of the first biomedical laser. The processor is further configured to analyze the measured light output properties and the operational response, and trigger an action based on the analysis.

Optionally, the light sensing arrangement comprises at least one of: a first light sensor adapted to be arranged in the laser component module, a second light sensor adapted to be arranged in proximity of the light guide, a third light sensor adapted to be arranged at an end of the light guide and a fourth light sensor adapted to be arranged inside of the optical head. The light sensing arrangement may comprise also any two or three of these sensors, for example (as described above) a first sensor and a second sensor, a first sensor and a third sensor, a first sensor and a fourth sensor, a second sensor and third sensor, a second sensor and a fourth sensor or a third sensor and a fourth sensor. Likewise, it may comprise a first, second and third sensor; a first, second and fourth sensor; a first, third and fourth sensor; or a second, third and fourth sensor. In an embodiment, the first light sensor, the second light sensor, the third light sensor and the fourth sensor are same kind of sensor. For example, the first light sensor, the second light sensor, the third light sensor and the fourth light sensor may be configured to measure the intensity of emitted laser light. In such example, the first light sensor, the second light sensor, the third light sensor and the fourth light sensor may be a photoresistor, a photo diode, or the like. In another embodiment, the first light sensor, the second light sensor, the third light sensor and the fourth light sensor are a combination of different kinds of sensors. For example, the first light sensor and the second light sensor may be configured to measure the intensity of the emitted laser light and the third light sensor may be configured to measure the wavelength of the emitted laser light. In such example, the first sensor and the second sensor may be a photoresistor, a photo diode, or the like, and the third sensor may be a wavemeter or a spectrometer. Optionally, the first light sensor is detachably coupled inside the light coupling arrangement. Additionally, the second light sensor may be affixed to the light guide. More optionally, the third light sensor may be placed such that the emitted laser light is incident upon light-sensitive elements of the third light sensor. More optionally, the fourth light sensor may be placed such that it can measure light inside of the optical head.

In an embodiment, the light sensing arrangement measures intensity of the ambient light. It is to be understood that based on the measured intensity of the ambient light, light output properties of the first biomedical laser is also determined by the light sensing arrangement. Furthermore, based on the measured intensity of the ambient light, the light sensing arrangement transmits a signal, indicative of the leakage of the emitted laser light, to the processor. Thereafter, the processor analyses the measured the light output properties and the operational response and triggers an action based on the analysis. For example, the action may be initiating a maintenance procedure for the first biomedical laser.

Optionally, the system comprises a server arrangement. Specifically, the server arrangement is communicably coupled to a communication module of the processor via a network. According to an embodiment, the server arrangement is configured to analyze the measured light output properties and the operational response and to trigger an action based on the analysis. Furthermore, the network may be wired, wireless or a combination thereof. Examples of the network include, but are not limited to, Local Area Networks (LANs), Wide Area Networks (WANs), radio network, Internet, radio networks, and telecommunication networks. Specifically, the communication module is configured to transmit the measured operational response and the light output properties to the server arrangement. More specifically, the server arrangement may be hardware, software, firmware or any combination of these. In an embodiment, the server arrangement comprises a database configured to store the measured operational response and the light output properties. For example, the database may store the measured power usage or the measured wavelength of the first biomedical laser.

According to a further embodiment, a person using the system can be identified for example by user name and password or a smart card. The database of the server system can be configured to have a profile data of the person such as what treatment modalities the person is allowed to have access and use (for example based on specialty of a doctor operating the system). Further, a profile data of the person can be extracted and the first set of operational parameters can be provided based on the profile data.

According to an additional embodiment, a location of the biomedical laser can be obtained. The location can be obtained by Internet Protocol (IP) address look up i.e. determining based on the IP address geo location. Alternatively the location can be obtained by registering the location in a database of with identification (ID) and other info related to the biomedical lasers. For example, ID of the biomedical laser can be associated with sales data or installation data comprising address, room etc. of the biomedical laser. The location information can be used to extract a setting data associated with the biomedical laser and the location. The setting data can be for example that a biomedical laser in non hospital environment cannot be used for medical purposes. The setting data can be used to provide the first set of operational parameters (also the second set if the device is moved).

According to an additional embodiment, product information can be obtained. The product information can be for example scanned information related to drug used by a patient. The scanned information can be a bar code of the medicine package or a RFID (radio frequency identifier) of the package. Additionally, product information can be related to any exchangeable part or additional part of the biomedical laser such as used optical head. The product information may be used to extract from a database an operational data associated with the product information and providing a first set of operational parameters based on the operational data. As an example, if the drug used by patient limits the range of power and wavelengths which can be used to treatments, that product information (of the used drug) can trigger a new set of operational parameters for the biomedical laser. Alternatively when for example a new configuration of the biomedical laser (obtaining a new set of product information) is obtained, this can be used to further extract a second set of operational data and further provide a second set of operational parameters to the biomedical laser.

Additionally ambient temperature around the biomedical laser can be obtained using a temperature sensor or external source (such as room temperature sensor of office/home automation system). The biomedical laser might have a range of approved ambient temperature ranges. The approved ambient temperature ranges are typically defined in the biomedical laser acceptance specification. The ambient temperature can be used as an additional parameters to determine treatment modalities. As an example if the ambient temperature is outside of safe operational margin the treatment modalities might be changed to prohibit usage of the biomedical laser totally or for certain treatments. Term ambient temperature can be understood broadly to refer to external or internal temperatures of the biomedical laser.

### DETAILED DESCRIPTION OF THE DRAWINGS

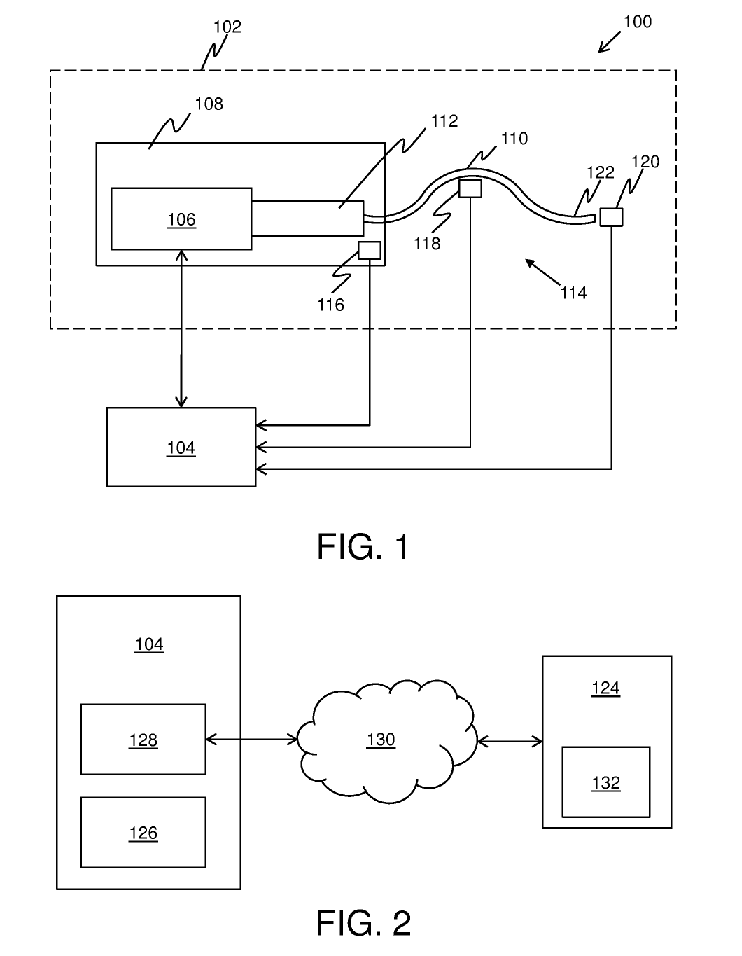

Referring to FIG. 1, there is shown a schematic illustration of a system 100 for configuring a first biomedical laser 102, in accordance with an embodiment of the present disclosure. The system 100 comprises a processor 104 coupled to the first biomedical laser 102. As shown, the first biomedical laser 102 comprises a laser light emitter 106 arranged in a laser component module 108. The first biomedical laser 102 further comprises a light guide 110 optically coupled to the laser light emitter 106 via a light coupling arrangement 112. The processor 104 is coupled with the first biomedical laser 102. The processor 104 is configured to provide a first set of operational parameters. Using the first set of operational parameters, the processor 104 operates the first biomedical laser 102. Furthermore, the processor 104 is configured to measure an operational response of the first biomedical laser 102. As shown, the system 100 further comprises a light sensing arrangement 114 communicably coupled to the processor 104, the light sensing arrangement 114 being configured to measure a light output properties of the first biomedical laser 102. In the illustrated embodiment, the light sensing arrangement 114 comprises a first light sensor 116, a second light sensor 118 and a third light sensor 120. The first light sensor 116 is adapted to be arranged in the laser component module 108. The first light sensor 116 is configured to measure the light output properties inside the laser component module 108. The second light sensor 118 is adapted to be arranged in proximity of the light guide 110. Therefore, the second light sensor 118 is configured to measure the light output properties, for example the energy of emitted laser light, in proximity of the light guide 110. The third light sensor 120 is adapted to be arranged at an end of the light guide 122. Therefore, the third light sensor 120 is configured to measure light output properties, for example, wavelength of the emitted laser light, at the end of the light guide 122. The processor 104 further analyzes the measured light output properties and the operational response. Thereafter, the processor 104 is configured to trigger an action based on the analysis.

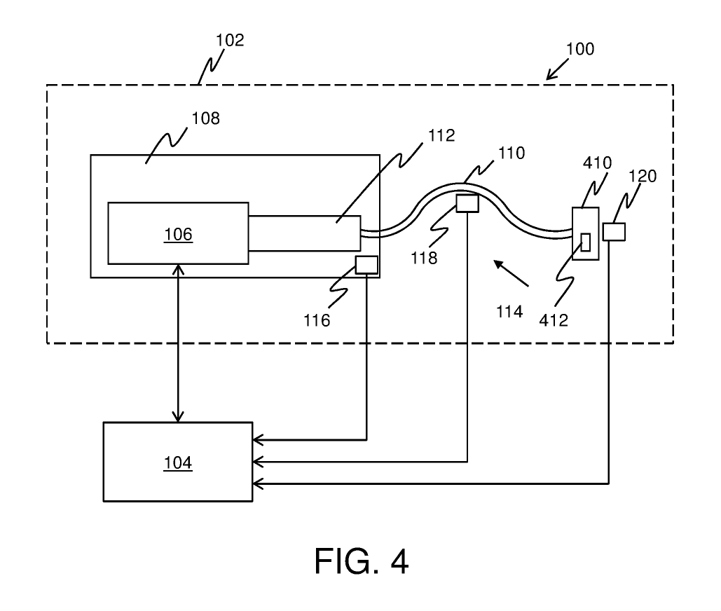

FIG. 4, illustrates another system 100 that comprises a fourth sensor 412. The fourth sensor is arranged inside of an optical head 410. The optical head 410 can be used to modify light from the laser light emitter 106 for example with a lenses system, collimators, splitters, actuating prims/mirrors used in scanning function etc. The processor 104 can be configured to control the optical head. Controlling of the optical head can be considered to be an operational parameter. The fourth sensor 412 can be used for example to detect operation of the scanning function.

Referring to FIG. 2, illustrated is a schematic block diagram of the processor 104 in communication with a server arrangement 124, in accordance with an embodiment of the present disclosure. The processor 104 comprises a memory module 126 and a communication module 128. The memory module 126 may optionally be configured to store the measured operational response and the light output properties. As shown, the communication module 128 of the processor 104 is communicably coupled to the server arrangement 124 via a network 130 to transmit the measured operational response and the light output properties to the server arrangement 124. The server arrangement 124 further comprises a database 132 to store the measured operational response and the light output properties received from the processor 104. The server arrangement 124 further comprises an analysis module for analyzing the measured operational response and the light output properties. The analysis can be used to trigger an action or to derive new set of operational parameters.

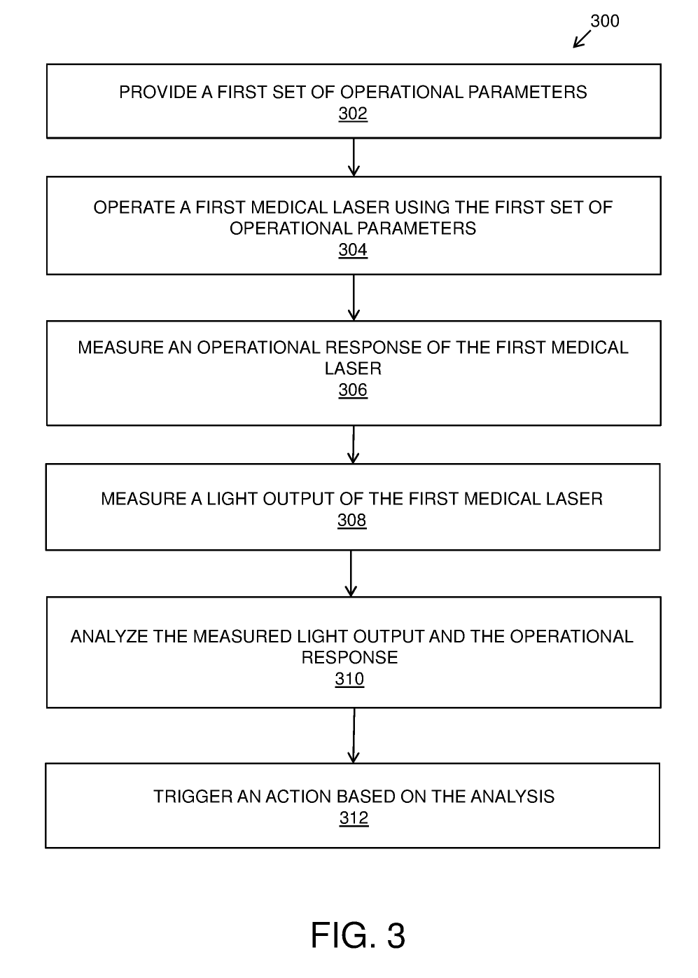

Referring to FIG. 3, illustrated are steps of a method 300 for configuring a first biomedical laser, in accordance with an embodiment of the present disclosure. At step 302, a first set of operational parameters are provided. At step 304, the first biomedical laser is operated using the first set of operational parameters. At step 306, an operational response of the first biomedical laser is measured. At step 308, a light output properties of the first biomedical laser is measured. At step 310, the measured light output properties and the operational response are analyzed. At step 312, an action is triggered, based on the analysis.

The steps 302 to 312 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method for configuring a first biomedical laser, the method comprising
providing a first set of operational parameters;
operating the first biomedical laser using the first set of operational parameters;
measuring an operational response of the first biomedical laser;
measuring a light output properties of the first biomedical laser;
analyzing the measured light output properties and the operational response; and
triggering an action based on the analysis.

2. A method according to claim 1, wherein the first set of operational parameters comprises at least one of a target wavelength of the first biomedical laser, a target energy output of the first biomedical laser and an operational mode selected from a group of a treatment modality, biomedical activation and measurement process.

3. A method according to claim 1, wherein measuring the light output properties of the first biomedical laser comprises measuring at least one of a wavelength of emitted laser light, a spectrum of emitted laser light, an energy of the emitted laser light, a pulse energy of the emitted laser light, an optical power of the emitted laser light, and a pulse form factor of the emitted laser light.

4. A method according to claim 1, wherein measuring the light output properties is carried out by arranging at least one of a first light sensor in a laser component module, a second light sensor in proximity of a light guide, a third light sensor at an end of the light guide and a fourth light sensor in an optical head.

5. A method according to claim 1, wherein measuring the operational response of the first biomedical laser comprises measuring at least one of voltage transient as a function of time, current transient as a function of time, temperature of a laser light emitter and power usage of the first biomedical laser during operation.

6. A method according to claim 1, wherein the analysis comprises comparing the measured operational response and the light output properties with a first set of reference values.

7. A method according to claim 6, wherein the first set of reference values comprises at least one of a target wavelength, a target energy output and a pulse form factor.

8. A method according to claim 1, wherein the action is deriving a second set of operational parameters based on the analysis and providing the second set of operational parameters.

9. A method according to claim 8, wherein the second set of operational parameters comprises operational parameters for a different treatment modality, parameters for biomedical activation or parameters for measurement process.

10. A method according to claim 1, wherein the action is initiating a maintenance procedure for the first biomedical laser.

11. A method according to claim 1, wherein the method further comprises transmitting the measured operational response and the light output properties to a server arrangement via a network.

12. A method according to claim 11, wherein the analyzing the measured light output properties and the operational response is carried out in the server arrangement.

13. A method according to claim 1, further comprising
storing the measured operational response and the light output properties;
basing a second set of reference values on the stored measured operational response and the light output properties; and
using the second set of reference values for configuring a second biomedical laser.

14. A method according to claim 13, wherein storing the measured operational response and the light output properties comprises storing the measured operational response and the light output properties at a memory unit of a processor coupled to the first biomedical laser.

15. A method according to claim 13, wherein storing the measured operational response and the light output properties comprises
transmitting the measured operational response and the light output properties to the server arrangement via a network; and
storing the measured operational response and the light output properties at a database of the server arrangement.

16. Method according claim 1, wherein the method further comprises identifying a person, extracting from the database a profile data of the person and providing the first set of operational parameters based on the profile data.

17. Method according to claim 1, wherein the method further comprises obtaining a location of the biomedical laser, extracting from the database a setting data of the associated with the biomedical laser and the location and providing the first set of operational parameters based on the setting data.

18. Method according to claim 1, wherein the method further comprises obtaining product information, extracting from the database an operational data associated with the product information and the biomedical laser and providing the first set of operational parameters based on the operational data.

19. A system for configuring a first biomedical laser, the system comprising
a processor coupled to the first biomedical laser, the processor being configured to
provide a first set of operational parameters;
operate the first biomedical laser using the first set of operational parameters;
measure an operational response of the first biomedical laser; and
a light sensing arrangement communicably coupled to the processor, the light sensing arrangement being configured to measure a light output properties of the first biomedical laser;
wherein the system is further configured to analyze the measured light output properties and the operational response, and to trigger an action based on the analysis.

20. A system according to claim 19, wherein the first biomedical laser comprises
a laser light emitter arranged in a laser component module; and
a light guide optically coupled to the laser light emitter via a light coupling arrangement.

21. A system according to claim 19, wherein the light sensing arrangement comprises at least one of
a first light sensor adapted to be arranged in the laser component module;
a second light sensor adapted to be arranged in proximity of the light guide;
a third light sensor adapted to be arranged at an end of the light guide and;
a fourth light sensor adapted to be arranged inside of the optical head.

22. A system according to claim 19, wherein the processor comprises a memory module, the memory module being configured to store the measured operational response and the light output properties.

23. A system according to claim 19, further comprising a server arrangement communicably coupled to a communication module of the processor via a network.

24. A system according to claim 23, wherein the server arrangement is configured to analyze the measured light output properties and the operational response and to trigger an action based on the analysis.

25. A system according to claim 23, wherein the server arrangement comprises a database configured to store the measured operational response and the light output properties.
